(19) United States
(12) Patent Application Publication
(10) Pub. No.: US 2019/0022328 A1
(43) Pub. Date: Jan. 24, 2019

(54) DOSAGE MEASUREMENT SYSTEM IN A PEN BUTTON

(71) Applicant: Verily Life Sciences LLC, Mountain View, CA (US)

(72) Inventors: Brett Schleicher, San Francisco, CA (US); Benjamin Krasnow, Redwood City, CA (US); Russell Mirov, Los Altos, CA (US); Adam Reich, Oakland, CA (US); John Redmond, San Francisco, CA (US)

(21) Appl. No.: 16/040,345

(22) Filed: Jul. 19, 2018

Related U.S. Application Data
(60) Provisional application No. 62/535,759, filed on Jul. 21, 2017.

(51) Int. Cl. A61M 5/315; A61M 5/31; A61M 5/34

(52) U.S. Cl. CPC A61M 5/31551; A61M 5/3146; A61M 5/24; A61M 5/31556; A61M 5/31546; A61M 5/347

(57) ABSTRACT

An apparatus includes a pen body of a drug injection pen including a dosage injection mechanism that produces a rotational motion when the drug injection pen dispenses a fluid. The apparatus also includes a button housing with at least part of a dosage measurement system disposed within the button housing. At least part of the dosage measurement system is coupled to receive the rotational motion from the dosage injection mechanism. The dosage measurement system includes one or more sensors positioned to output a signal in response to the rotational motion when the drug injection pen dispenses a fluid, and a controller coupled to the one or more sensors to receive the signal. A drug delivery control wheel of the drug injection pen is disposed between the body of the drug injection pen and the at least part of the dosage measurement system disposed in the button housing.

### REFERENCE TO RELATED APPLICATION

This application claims the benefit of U.S. Application No. 62/535,759, filed on Jul. 21, 2017, the contents of which are incorporated herein by reference.

### TECHNICAL FIELD

This disclosure relates generally to drug injection and in particular but not exclusively, relates to tracking injection quantities.

### BACKGROUND INFORMATION

Measuring the quantity and recording the timing of a drug's administration is an integral part of many disease treatments. For many treatments, to achieve the best therapeutic effect, specific quantities of a drug may need to be injected at specific times of day. For example, individuals suffering from diabetes may be required to inject themselves regularly throughout the day in response to measurements of their blood glucose. The frequency and volume of insulin injections must be carefully tracked and controlled to keep the patient's blood glucose level within a healthy range.

Currently, there are a limited number of methods or devices capable of tracking drug administration without requiring the user to manually measure and record the volume, date, and time. A variety of glucose injection syringes/pens have been developed, but there is much room for significant advancement in the technology in order to reduce the size, lower the cost, enhance the functionality, and improve the accuracy. Thus, the current technology may not be an ideal long-term solution. For example, current insulin pens are often disposable, but do not include dosage tracking. A smaller portion of the market is composed of reusable pens which are more expensive, and still do not include accurate dosage-tracking capabilities.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified. The drawings are not necessarily to scale, emphasis instead being placed upon illustrating the principles being described.

FIG. 1 illustrates an injection pen system, in accordance with an embodiment of the disclosure.

FIG. 2A illustrates part of an injection pen and a pen button, including a dosage measurement system, in accordance with an embodiment of the disclosure.

FIG. 2B illustrates a cross section of the pen button and injection pen of FIG. 2A, in accordance with an embodiment of the disclosure.

FIG. 2C illustrates the pen button of FIG. 2A inserted into the pen body, in accordance with an embodiment of the disclosure.

FIG. 2D illustrates a cross section of the pen button and injection pen of FIG. 2C, in accordance with an embodiment of the disclosure.

FIG. 2E illustrates an exploded view of the pen button of FIG. 2A, in accordance with an embodiment of the disclosure.

FIG. 3A illustrates a pen button including a dosage measurement system, in accordance with an embodiment of the disclosure.

FIG. 3B illustrates the pen button of FIG. 3A with the button housing removed, in accordance with an embodiment of the disclosure.

FIG. 3C illustrates a circuit board, from the pen button of FIGS. 3A and 3B, for a strain based dosage measurement system, in accordance with an embodiment of the disclosure.

FIG. 3D illustrates a cogwheel that imparts a strain on the circuit board in FIG. 3C, in accordance with an embodiment of the disclosure.

FIG. 3E illustrates a circuit which may be used to implement part of the circuit board of FIG. 3C, in accordance with an embodiment of the disclosure.

FIG. 4A illustrates a strain-based dosage measurement system, in accordance with an embodiment of the disclosure.

FIG. 4B illustrates another strain-based dosage measurement system, in accordance with an embodiment of the disclosure.

FIG. 4C illustrates an electrical output from the strain-based dosage measurement system of either FIG. 4A or 4B, in accordance with an embodiment of the disclosure.

FIG. 5A illustrates an exploded view of a pen button including a dosage measurement system, in accordance with an embodiment of the disclosure.

FIG. 5B illustrates an assembled view of the pen button of FIG. 5A with the housing cut away, in accordance with an embodiment of the disclosure.

FIG. 5C illustrates an encoder which may be included in the pen button of FIG. 5A, in accordance with an embodiment of the disclosure.

FIG. 6 illustrates a method of dosage measurement, in accordance with an embodiment of the disclosure.

FIG. 7 illustrates a method of fabricating a drug injection pen including a button to measure a dosage dispensed, in accordance with an embodiment of the disclosure.

FIGS. 8A-8B illustrate an exploded view of the pen button, in accordance with an embodiment of the disclosure.

### DETAILED DESCRIPTION

Embodiments of an apparatus and method for dosage measurement from a drug injection pen are described herein. In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The present disclosure is directed at systems and methods for measuring and tracking a quantity of fluid dispensed from a drug injection pen (e.g., an insulin pen, or other self-administered medication). Currently, there are a limited number of viable options to accurately track the quantity of fluid dispensed from injection pens. Often dosage is correlated with how much medication the user selects (dials) to inject. Unfortunately, this is may not be the same thing as the quantity actually injected, since the user can dial back the dosage selected. Further systems disclosed herein measure the actual rotation of the dosage injection mechanism (e.g., the “lead screw” or “plunger” in the pen). This method removes noise that may otherwise find its way into the measurement. For example, other methods may use acoustics to determine the dosage selected, but may register a dose when the pen bumps into another object. Moreover, the systems disclosed here are either built into the injection pen itself, or a button that attaches to the pen, so the user does not need to worry about losing the device or having it fall off the pen.

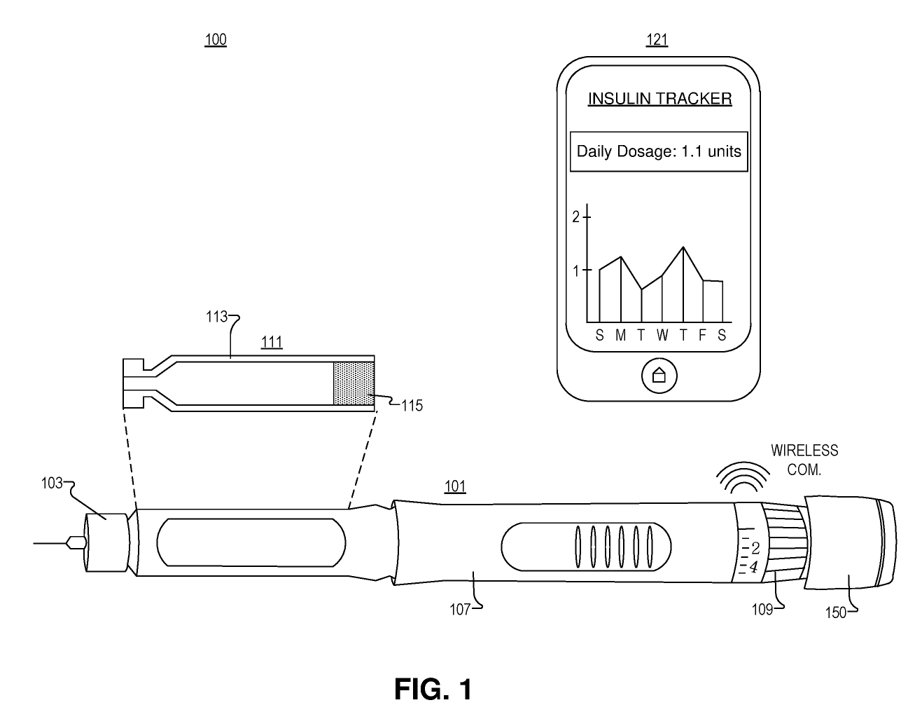

FIG. 1 illustrates an injection pen system 100, in accordance with an embodiment of the disclosure. Pen system 100 includes injection pen 101, drug cartridge 111, and processing device 121 (e.g., a smart phone).

Drug cartridge 111 includes cartridge body 113, and plunger head 115. In the depicted embodiment, plunger head 115 starts near the rear of drug cartridge 111 and is pushed forward in drug cartridge 111 (with a dosage injection mechanism disposed in injection pen 101). This forces medication/fluid out of the narrow end of drug cartridge 111 when a user chooses to dispense a fluid. In one embodiment, cartridge body 113 includes borosilicate glass.

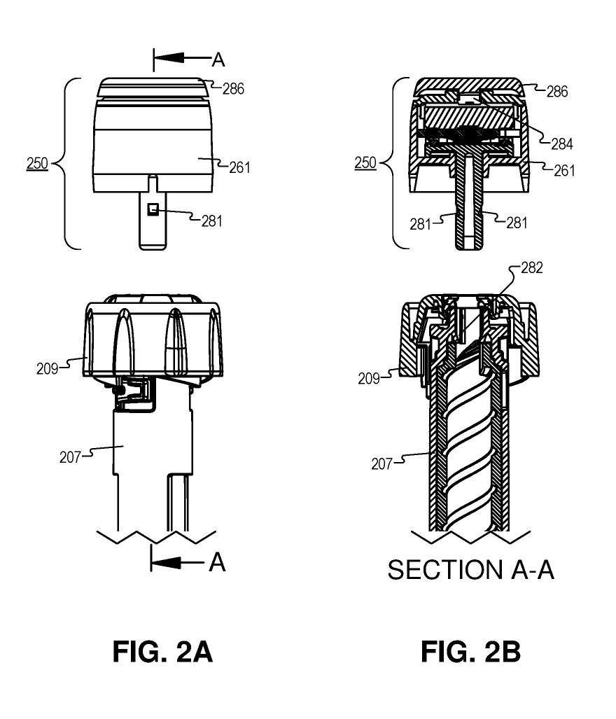

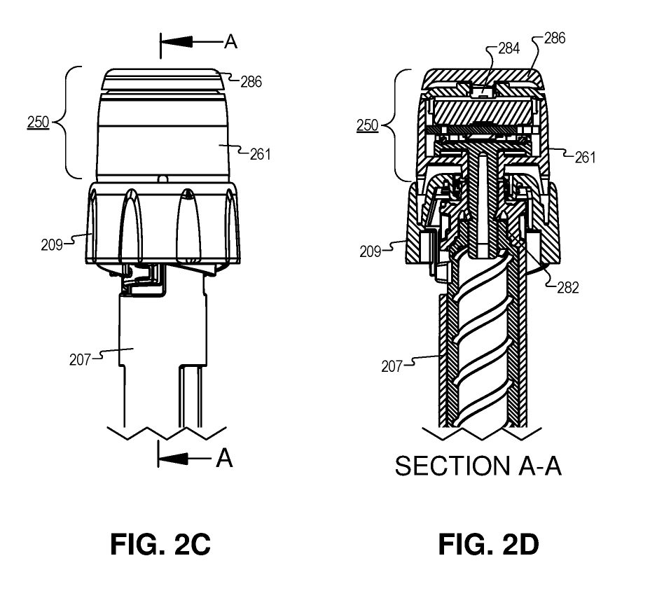

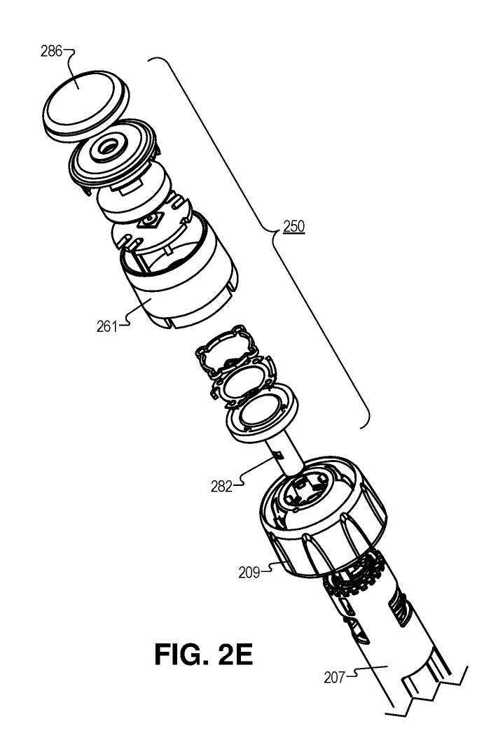

Injection pen 101 is a hand-held device and includes needle 103, body/housing 107 (including a dosage injection mechanism to push in plunger head 115 and extract fluid from drug cartridge 111), and drug delivery control wheel 109 (twist wheel 109 to “click” select the dosage), and pen button 150 (push button 109 to dispense the selected quantity of the fluid from cartridge 111). It is appreciated that pen button 150 may include a dosage measurement system (see e.g., FIGS. 2A-5C). As shown, housing 107 is configured to accept cartridge 111: cartridge 111 may be disposed in an insert which screws/snaps onto the bulk of housing 107. However, as one of ordinary skill in the art will appreciate, injection pen 101 can take other configurations and have other components.

As stated, injection pen 101 includes a housing/body 107 shaped to accept a cartridge containing a fluid, and also includes a dosage injection mechanism positioned in the housing 107 to produce a rotational motion and force the fluid out of the cartridge when the drug injection pen 101 dispenses the fluid. A dosage measurement system is also disposed in the pen (e.g., in button 150 or elsewhere in pen body 107) to receive a rotational motion from the dosage injection mechanism. The dosage measurement system may measure a strain induced in a portion of the dosage measurement system by the rotational motion, and the dosage measurement system outputs a signal indicative of the strain when the drug injection pen 101 dispenses the fluid.

A controller is also disposed in drug injection pen 101, and is coupled to the dosage measurement system. The controller includes logic that when executed by the controller causes the controller to record the electrical signal output from the dosage measurement system when (not before or after) drug injection pen 101 dispenses the fluid. One of ordinary skill in the art will appreciate that the controller may be static (e.g., have logic in hardware), or dynamic (e.g., have programmable memory that can receive updates). In some embodiments, the controller may register the electrical signal output from the dosage measurement system as an injection event of the fluid, and the controller may calculate a quantity of the fluid dispensed based, at least in part, on a number of the injection events of the fluid registered by the controller. It is appreciated that this circuitry, which will be described in greater detail in connection with other figures, may be disposed anywhere in drug injection pen 101 (e.g., in body/housing 107 or pen button 150), and in some instances, logic may be distributed across multiple devices.

Processing device 121 (e.g., a smartphone, tablet, general purpose computer, distributed system, servers connect to the internet, or the like) may be coupled to receive dosage data from injection pen 101 to store/analyze this data. For instance, in the depicted embodiment, processing device 221 is a smartphone, and the smartphone has an application running recording how much insulin has been spent from pen 101. Moreover, the application is plotting how much insulin has been injected by the user over the past week. In this embodiment, a power source is electrically coupled to the controller in injection pen 101, and a transceiver is electrically coupled to the controller to send and receive data to/from processing device 121. Here, data includes information indicative of a quantity of the fluid dispensed. Transceiver may include Bluetooth, RFID, or other wireless communications technologies.

FIG. 2A illustrates part (body/housing 107) of an injection pen, and pen button 250, including a dosage measurement system, in accordance with an embodiment of the disclosure. It is appreciated that the components in FIG. 2A may be included in the injection pen 100 of FIG. 1. As shown pen button 250 is fabricated to be inserted into the proximal end of the injection pen (opposite a dispensing end of the injection pen). Pen button 250 includes a pair of notches 281, cut into a shaft/column protruding from pen button 250, which clip into the injection pen. It is appreciated that the pen button housing 261 contains the dosage measurement system including electronics to measure a rotational motion of the dosage injection mechanism of the pen.

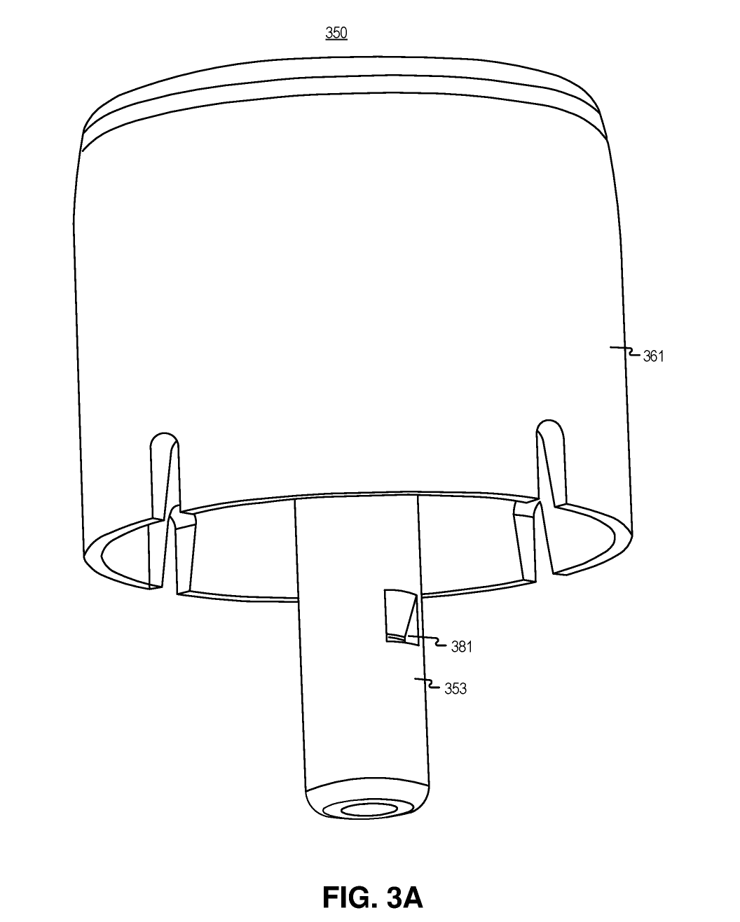

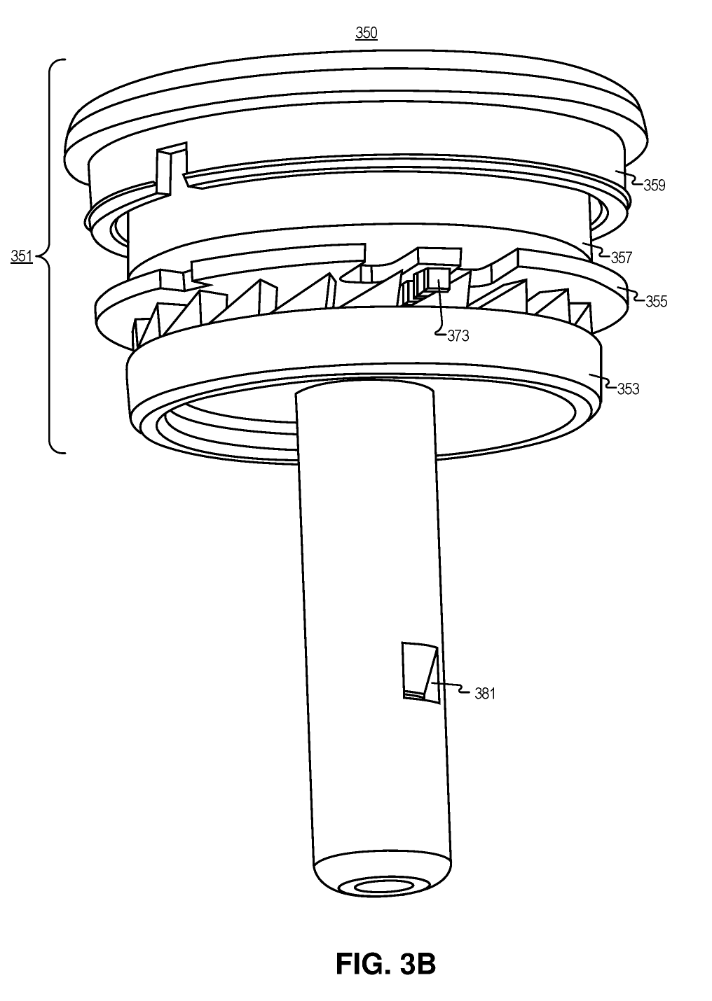

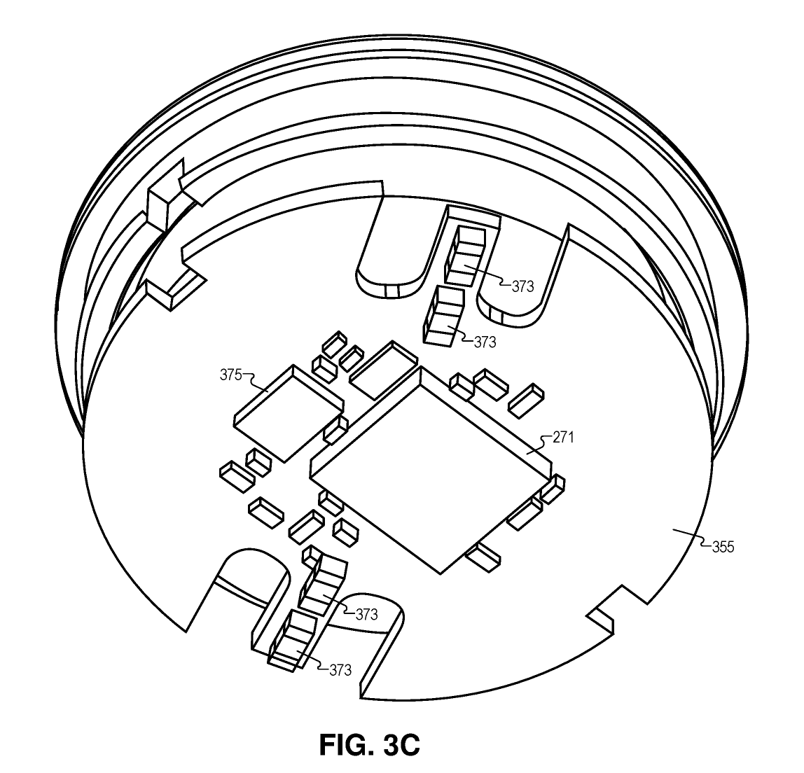

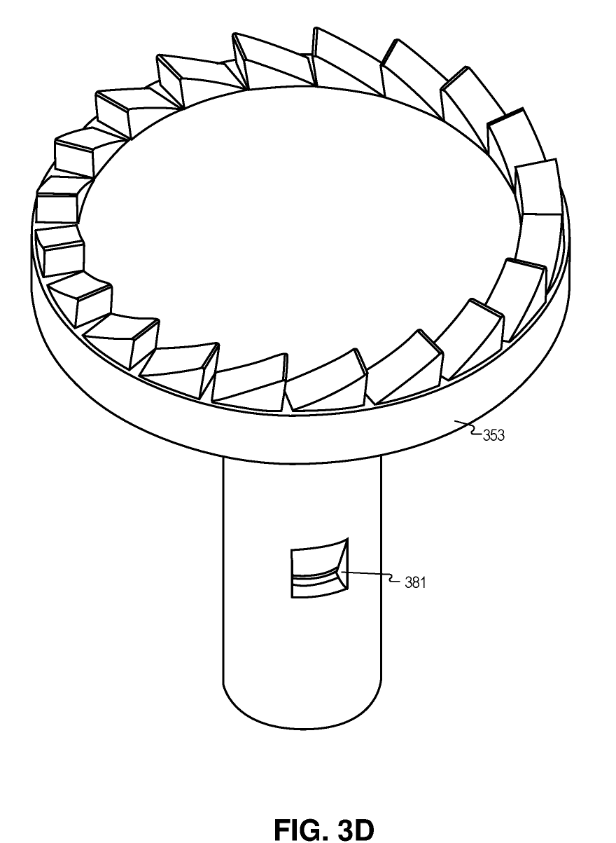

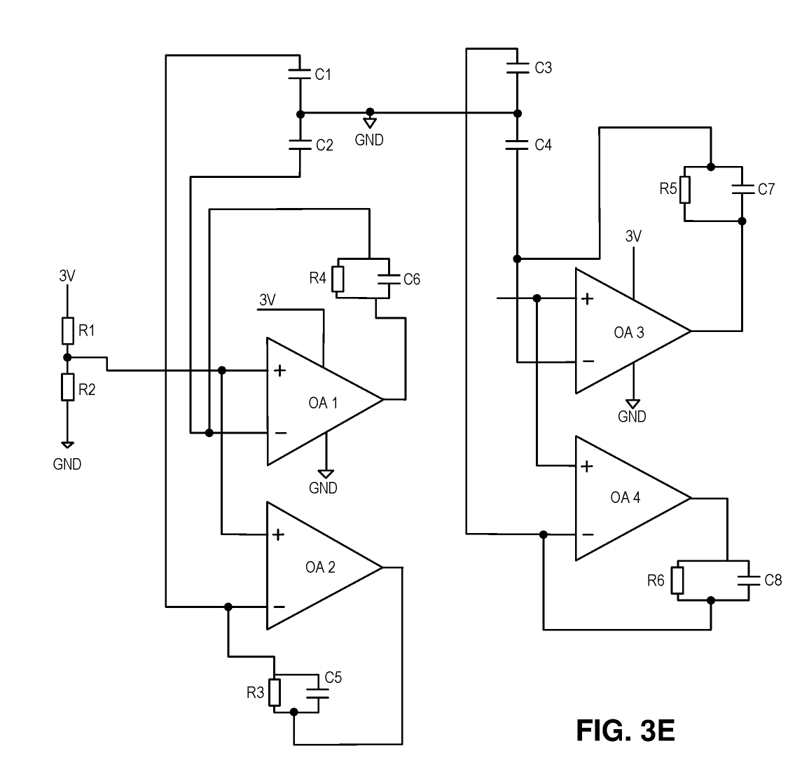

FIG. 2B illustrates a cross section of the pen button and injection pen of FIG. 2A, in accordance with an embodiment of the disclosure. As depicted, pair of notches 281 are cut into the shaft (e.g., column of toothed gear 353 or the like, see infra FIG. 3D), protruding from pen button 250. A pair of locking tabs 282 are disposed in the pen housing 107 that fit into notches 281, and provide both axial restraint (so pen button 250 doesn't fall out), and also rotational locking so that pen button 250 experiences relative rotation between shafts of the dosage injection mechanism when the pen is dispensing a dose. The body of pen button 250 is rotationally locked to the drug delivery control wheel 209 (the largest diameter part in FIG. 2B) via four slots.

FIG. 2C illustrates pen button 250 of FIG. 2A inserted into pen body 207, in accordance with an embodiment of the disclosure. As shown pen button 250 clips into the proximal end of the injection pen, so that drug delivery control wheel 209 is disposed between pen button 250 and pen housing 207. In other words, a component in the dosage measurement system of the button irremovably clips to the dosage injection mechanism in the drug injection pen.

FIG. 2D illustrates a cross section of the pen button 250 and injection pen of FIG. 2C, in accordance with an embodiment of the disclosure. As shown, pair of locking tabs 282 fit into notches 281 to hold pen button 250 in place. In some embodiments, pen button 250 can be fabricated separately from the rest of the injection pen and then “snap” into the injection pen in assembly. Thus, the pen assembly process merely involves rotational alignment of the button 250 notches with the pins in the drug delivery control wheel 209, and alignment of the notches 281 in the button shaft to locking tabs 282. Then, pen button 250 is pressed straight into the pen. Locking tabs 282 are tapered so that they allow insertion, but not removal.

An additional unique aspect of an embodiment is that pen button 250 spins when the pen dispenses fluid. In the depicted embodiment, pen button 250 rotates along with drug delivery control wheel 209 when the pen is dispensing a dose. The user's thumb does not interfere with this rotation, so thrust bearing 284 and spinner 286 are disposed on top of pen button 250. Thus all electronics in pen button 250/dosage measurement system spin when the injection pen dispenses fluid, but the user's thumb and fingers do not prevent dispensing of the fluid. In other words, a first portion of the button housing (e.g., the sides of the button housing 261 and the internal electronics) is coupled to rotate around a longitudinal axis of the drug injection pen when attached to the dosage injection mechanism, and a second portion of the button housing (e.g., spinner 286) is coupled to rotate independently from the first portion.

FIG. 2E illustrates an exploded view of the pen button of FIG. 2A, in accordance with an embodiment of the disclosure. As shown, pen button 250 includes a number of components (which will be described in greater detail later below) that are stacked in a layered configuration in the pen button 250. For example, a circuit board containing strain measurement circuitry may be sandwiched between a cogwheel to impart strain and a power source (e.g., battery, capacitive storage, inductive charging loop, etc.).

FIG. 3A illustrates a pen button 350—which may be the pen button 150 of FIG. 1—including a dosage measurement system, in accordance with an embodiment of the disclosure. A pen button housing 361 is shaped to attach to a proximal end of the drug injection pen (e.g., drug injection pen 101) opposite a dispensing end of the drug injection pen. As stated above, it is appreciated that pen button 350 may snap into a commercially available drug injection pen, or may be designed to be built into a custom pen. The bottom of a toothed gear 353 is visible from under button housing 361.

FIG. 3B illustrates the pen button 350 of FIG. 2A with button housing 361 removed, in accordance with an embodiment of the disclosure. As shown, a dosage measurement system 351 is disposed at least in part in button housing 361. Dosage measurement system 351 includes a toothed gear 353, and circuit board 355—with one or more strain sensors 373 coupled to a controller (see FIG. 3C, controller 371). Dosage measurement system 351 is positioned to monitor a rotational motion of the pen's dosage injection mechanism (e.g., one or more rotating hollow columns, or lead screws, disposed within the drug injection pen housing) when the drug injection pen dispenses the fluid. This is achieved by the columnar portion of toothed gear 353 attaching to one or more of the rotating columns (see e.g., FIGS. 2B and 2D, locking tabs 282 setting into notches 281) to rotate when the pen dispenses the fluid. When toothed gear 353 rotates relative to circuit board 355, one or more strain sensors 373 measure a strain imparted in circuit board 355, and output a signal to the controller. Thus, toothed gear 353 is coupled to the dosage injection mechanism to rotate when the drug injection pen dispenses the fluid, and the strain sensors 373 are positioned to be contacted by teeth in toothed gear 253 when toothed gear 353 rotates. In other words, dosage measurement system 351 includes one or more strain sensors 373 disposed on a flexible component (e.g., the protrusions from circuit board 355) of dosage measurement system 351 to measure the strain in the flexible components when the drug injection pen dispenses the fluid. It is appreciated that strain sensors 373 may include a capacitive strain sensor, a piezoelectric strain sensor, or a resistive strain sensor.

Also depicted is power source 357 (e.g., a battery or the like) coupled to the controller and disposed at least in part within the push-button housing. Underneath the top 359 of the button may also be a transceiver (e.g., blue tooth, RFID, or the like) coupled to the controller to send and receive data, a charging device (e.g., a metal coil coupled to power source 357 for inductive charging), or the like. The transceiver may be instructed by the controller to transmit data, including information indicative of the number of the injection events, to an external device (e.g., processing device 121 of FIG. 1).

FIG. 3C illustrates a circuit board 355, from the pen button of FIGS. 2A and 2B, for a strain based dosage measurement system 351, in accordance with an embodiment of the disclosure. As shown, circuit board 355 includes one or more strain sensors 373 that measure strain imparted on circuit board 355 when the teeth on toothed gear 353 cause circuit board 355 to deform. In other words, circuit board 355 includes flexible component (e.g., protrusions), and one or more strain sensors 373 are positioned on circuit board 355 to measure the strain in circuit board 355 when the toothed gear rotates relative to the circuit board 355. At least one strain sensor 373 is disposed on one or more protrusions from circuit board 355. In the depicted embodiment, four strain sensors 373 are coupled to controller 371, and controller 371 includes logic that when executed by controller 371 causes controller 371 to perform operations including recording the signal output from the dosage measurement system in response to the drug injection pen dispensing the fluid. Further, controller 371 may register the signal as an injection event of the fluid, and calculate a quantity of the fluid dispensed based, at least in part, on a number of the injection events registered by controller 371. It is appreciated that controller 371 may register the number of injection events in memory 375 which may include RAM, ROM, or the like. Moreover, other pieces of circuitry are disposed on the circuit board 355, such as a clock (e.g., oscillator), operational amplifiers (see e.g., FIG. 2E), and the like.

As shown, strain sensors 373 include capacitors that are positioned on portions of circuit board 355 which are cut away to create springy protruding sections. The outboard set of capacitors provide a mechanical interface with toothed gear 353, and deform circuit board 355 as each tooth is pushed past the capacitor. Having two capacitors for each spring section provides signal redundancy, and also a precise, easy-to-manufacture method to mechanically interface circuit board 355 with toothed gear 353. The radial (clock position) placement of the two circuit board 355 spring sections is 189 degrees apart, which allows one section to slip off a tooth while other section is mid-way up the tooth ramp for a tooth wheel with 20 teeth (e.g., toothed gear 353 depicted in FIG. 3D). Thus the capacitors are 180 degrees out-of-phase and provide resolution of 40 counts per rotation even though the tooth wheel has only 20 teeth.

As shown, strain sensors 373 may be a multi-layer ceramic capacitor (MLCC) that is soldered to a printed circuit board 355 (either very thin FR-4 composite, or Kapton) which is physically attached to a portion of the injection pen's dosage injection mechanism. However, one of ordinary skill in the art having the benefit of the present disclosure will appreciate that the “strain sensors” disclosed here are inclusive of devices that measure other physical quantities (e.g., stress, shear stress, acceleration, etc.) that can be correlated to strain. Also, strain sensors are not limited to capacitors, and may include accelerometers, MEMs beams, snaked wires, etc.

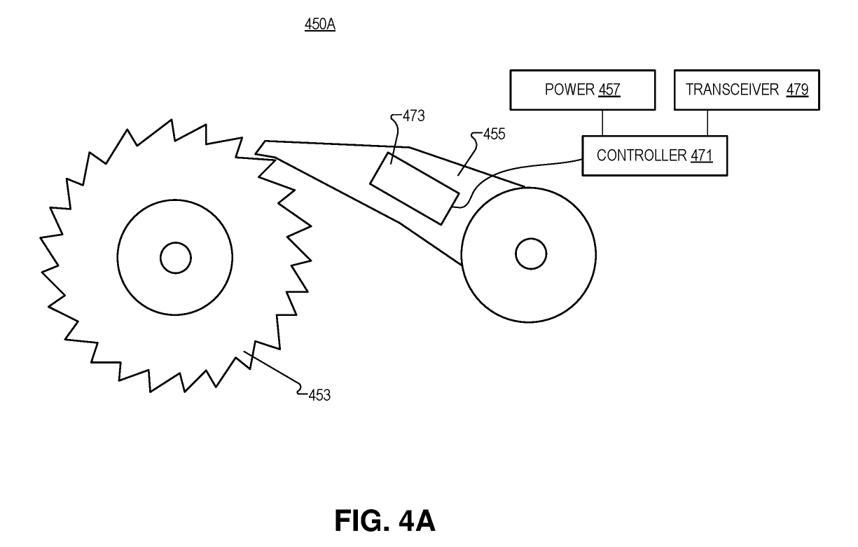

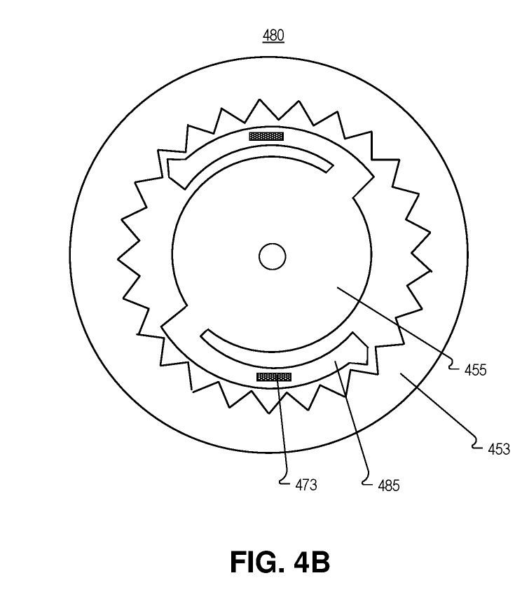

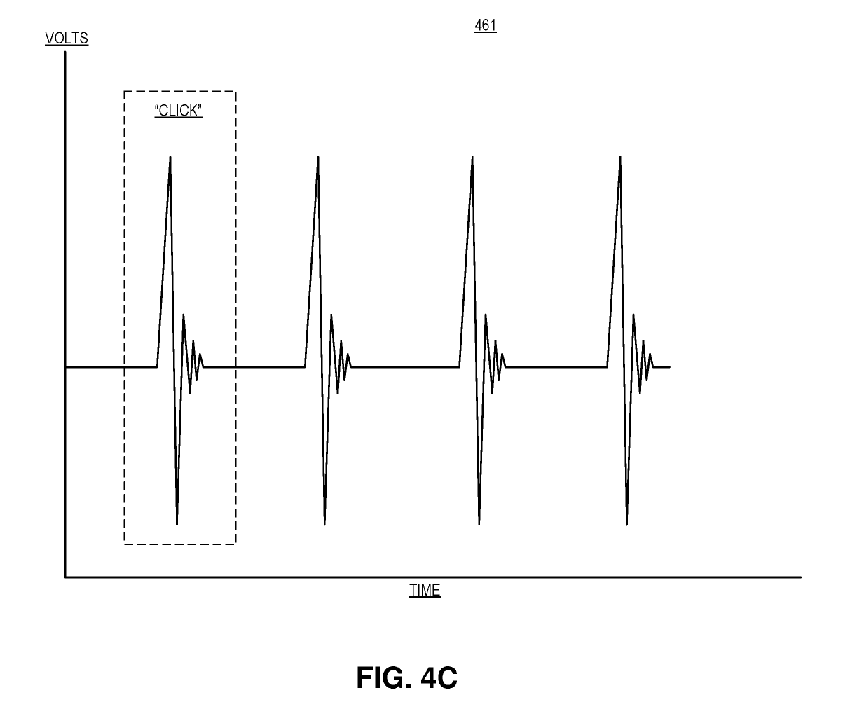

In the depicted embodiment, strain is measured in a portion (e.g., protrusions from circuit board 355 with “U”-shaped cut-aways on either side) of circuit board 355 that flexes or pivots during normal pen operation when dispensing medication. These flexes (mechanical strains) travel through the printed circuit board 355, and through the solder connections to the MLCC which measure the strain in circuit board 355 and solder. When the MLCC is charged with a bias voltage, the mechanical strain will cause the voltage to fluctuate (see e.g., FIG. 4C), which may be detected with an analog amplifier and microcontroller (see e.g., FIG. 3E). In several embodiments, strain gauges 373 may generate voltage spikes of 20 mV when they are attached to protrusions that flex when an injection pen's dispensing mechanism moves. The protrusion are dragged across a toothed surface which causes a repetitive mechanical strain for each tooth that is passed. Thus, by counting the voltage spikes, controller 371 can determine rotation distance to a precision determined by the tooth pitch.

FIG. 3D illustrates a toothed gear 353 that imparts the strain on the circuit board shown in FIG. 3C, in accordance with an embodiment of the disclosure. As shown, a columnar portion of toothed gear 353 is shaped to extend into, and attach to, a lead screw (e.g., part of the dispensing mechanism) to receive rotational motion. The teeth on toothed gear 353 extend outward from toothed gear 353 in a direction of the proximal end of the pen housing. However, in other embodiments they may extend out from the sides of toothed gear 353 (see infra FIG. 4A). While the teeth in the depicted embodiment are saw-tooth shaped to allow for one-way motion, in other embodiments the teeth may be rounded bumps to permit two-way motion. However, one of ordinary skill in the art having the benefit of the present disclosure will appreciate that the teeth may take any number of configurations, in accordance with the teachings of the present disclosure.

FIG. 3E illustrates a circuit which may be used implement part of the circuit board of FIG. 3C, in accordance with an embodiment of the disclosure. One of ordinary skill in the art having the benefit of the present disclosure will appreciate that there are many ways to implement similar strain based sensing circuits, and that pieces of circuitry may be substituted for other like parts, in accordance with the teachings of the present disclosure.

As stated above, strain sensors 373 may include four surface-mount capacitors (C1-C4) mounted on a circuit board (e.g., circuit board 355) in the mechanical CAD renderings in FIGS. 3A-3D. In the depicted embodiment, the capacitors are coupled to operational amplifiers (OAs 1-4), which output voltage signals (spikes) that are supplied to the controller (which may be a digital microcontroller). In the depicted embodiment, the raw voltage change from the capacitors due to mechanical strain is approximately 20 mV, which may not be high enough to be recorded by controller 371. Accordingly, the signal is amplified with the four operational amplifiers depicted, which are coupled to capacitors C1-C4. The output pulse of the operational amplifiers is approximately 2V. The operational amplifiers may be configured to be a standard inverting amplifier with the non-inverting input connected to a bias voltage which is approximately 90% of the supply voltage.

In the depicted embodiment, the operational amplifiers will serve their output to apply this bias voltage through a feedback resistor to the non-inverting input, which is connected to each sensor capacitor, and provides a constant bias voltage on the capacitor. Importantly, the circuit only consumes power in the operational amplifier itself, leakage through the sensor capacitors, and the voltage divider (R1 and R2) to create the bias voltage. Total power consumption for the circuit depicted may only be several microamps. The operational amplifiers are selected to be low-power, low-bandwidth, rail-to-rail components.

In some embodiments, three additional resistors may be used to create a Wheatstone bridge (a four resistor configuration that results in extremely accurate strain measurements). A benefit of using chip resistors instead of foil or silicon strain gages is that the resistance achieved in the thick-film resistors is much higher than what is possible with other gauges (generally limited to 1 kOhm), which permits much lower parasitic losses due to excitation current. In some bridge embodiments, the three resistors (that may not measure the strain) do not need to be thick-film-based.

FIG. 4A illustrates a strain-based dosage measurement system, in accordance with an embodiment of the disclosure. In the depicted embodiment, a pawl 455 and cogwheel 453 (e.g., a different embodiment of toothed gear 353) dosage measurement system 450A is employed. Pawl 455 and cogwheel 453 of dosage measurement system 450A may be included in the device depicted in FIG. 1. As shown, a circular center of cog 453 is disposed to engage with the dosage injection mechanism (e.g., with a columnar portion that extends into, or out of, the page in the Z-direction, and may couple to the dosage injection mechanism, see e.g., FIGS. 2A-2C) is disposed in the center of cogwheel 453, and the column may transfer rotational motion from the dosage injection mechanism to cogwheel 453. Thus, cogwheel 453 spins when a dosage of medication is dispensed. As shown, pawl 455 includes strain sensor 473 (e.g., capacitive devices or the like discussed above) electrically coupled to controller 471. Accordingly, when cogwheel 453 spins, teeth from cogwheel 453 pass under pawl 455. With every tooth that passes beneath pawl 455, pawl 455 is deformed and stain sensor 473 outputs a characteristic electrical single. In one embodiment, pawl 455 may be considered a “circuit board” since strain sensor 473 and other circuitry may be disposed on pawl 455. Strain sensor 473 may include a variety of transducers including a piezoelectric sensor, a strain gauge, a pressure sensor, a capacitive sensor, or the like. In some embodiments, transducer 471 may include a piezoelectric material coating pawl 455, or in some embodiments pawl 455 may be fabricated from a piezoelectric material (quartz, polytetrafluoroethylene, or the like).

Many medication injection pens (e.g., pen 101 of FIG. 1) make use of a plastic ratchet mechanism that ensures the rubber stopper only pushes medication out of the device. Thus, dosage tracking may be implemented with pawl 455 that drags along cogwheel 453. As cogwheel 453 turns, pawl 455 clicks into place past each tooth on cogwheel 453, preventing cogwheel 453 from turning backwards. The one-way rotational movement ensures that the medication is only pushed out of the device, and that the mechanism can never backtrack. As shown, to achieve dose measurement functionality a thin film of piezoelectric polymer (e.g., part of transducer 473) may be added to pawl 455. These polymer films, such as polyvinylidene fluoride (PVDF) are readily available and very low-cost. In many pens, pawl 455 may have dimensions of approximately 1×4 mm, and the entire face of the pawl 455 could be covered by a PVDF film 50 microns thick. However, as shown only part of pawl 455 (or a place with highest stress/strain) may be covered. Both surfaces of the film are commonly metalized with a physically-deposited electrode. Electrical attachments can be made with conductive adhesive to connect the film to a conventional printed circuit board. Each time pawl 455 clicks past a tooth on cogwheel 453, the sudden change in pawl curvature causes the piezoelectric film to produce a voltage spike (see e.g., FIG. 4C). Thus, the rotation of cogwheel 453 is measured in steps.

In other embodiments, pawl 455 geometry can be modified such that the pawl 455 allows cogwheel rotation in either direction, but still gives a characteristic “click” as pawl 455 slips past each cogwheel tooth. The effect is similar to turning a knob that has detents, such as a low/med/high fan selector knob. In this embodiment, pawls 455 can be spaced 90 degrees out of phase with each other, and will deliver alternating voltage pulses in a quadrature pattern, thus detecting rotation direction as well as amount.

FIG. 4B illustrates another strain-based dosage measurement system—with a different type of pawl and cogwheel configuration—in accordance with an embodiment of the disclosure. In the depicted embodiment, circuit board 455 is coupled to rotate in response to the rotational motion from the dosage injection mechanism, and circuit board 455 includes one or more protrusions 485 (pawls extending outward from circuit board 455) that are positioned to be contacted by teeth 453 (e.g., in a stationary cogwheel) when circuit board 455 rotates. In other words, in the depicted embodiment teeth 481 are stationary inside the drug injection pen while circuit board 455 rotates. As shown, protrusions 485 that extend from circuit board 455 partially encircle a main portion of circuit board 455 (e.g., protrusions 485 extend outward from, and encircle, circuit board 455), and one or more strain sensors 473 are disposed on the one or more protrusions 485 to measure strain in the one or more protrusions 485. It is appreciated that strain sensors 473 may be placed in locations of maximum deformation in order to achieve the strongest signal. Like the pawl and cogwheel of FIG. 4A, strain sensors may include thin polymer films deposited on the protrusions 485, or may be built into protrusions 485.

In one embodiment circuit board 455 may be a Kapton flex material, and a 1 uF capacitor—in the 0805 surface mounted device (SMD) size conforming to X7R specification—may be attached to circuit board 455 as strain sensors 473. The capacitor may be attached to the plastic pawl mechanism (protrusions 485) with a rigid adhesive (e.g., cyanoacrylate). However, in other embodiments, one or more strain sensors 473 are constructed within the circuit board 455. A DC bias voltage of 5V may be applied through a 1 MOhm resistor so that the voltage spikes generated by the mechanical strains can be detected without being unduly influenced by the bias supply. Flexing the capacitor without a bias voltage does not produce a voltage spike. One benefit of this device architecture is that the microcontroller and associated circuitry can be assembled onto the same flexible circuit board 455 that contains the sensor MLCC, and is also attached to the plastic target mechanism. Thus, assembly and manufacturing costs may be lowered. Furthermore, the shape of circuit board 455 can be chosen to enhance the mechanical strain experienced by the sensor MLCC while isolating the other electronic components. For example, the shape of the circuit board may look like an hourglass where one lobe is rigidly attached to the flexing plastic member, and the other lobe is free-floating or fixed to a non-bending portion and is relatively isolated from the bending.

As illustrated, circuit board 455 itself may be used as flapper sensor—positioned in such a way that the circuit board 455 edge is in contact with a radial or linear track of gear teeth. The circuit board (or more specifically protrusion 485) is flexed each time it is pushed past a tooth. Additionally, multiple flapper sensors could be integrated into circuit board 455. For example, flexible element(s) on the perimeter could encode rotational count against a set of fixed gear teeth 453 or spline elements. An inner track could encode the up and down motion against bosses mounted on a planar surface. Multiple perimeter sensors with simple alternation will likely debounce the noisy indications from each sensor.

FIG. 4C illustrates an electrical output from the strain-based dosage measurement system of either FIG. 4A or 4B, in accordance with an embodiment of the disclosure. As stated in connection with FIGS. 4A and 4B, every time the pawl passes over a tooth of the cogwheel, it outputs a characteristic electrical signal from the transducer(s). Here, this electrical output has been graphed with respect to voltage and time. As shown, every time the pawl passes over as tooth, the voltage spikes. Each of these clicks may be correlated to a quantity of fluid dispensed from the injection pen. The number of clicks may be stored and used to determine how much medication has been dispensed, in accordance with the teachings of the present disclosure. One of ordinary skill in the art having the benefit of the present disclosure will appreciate that other electrical signals (other than voltage with respect to time, e.g., current, capacitance or the like) may be used to accurately measure dosage.

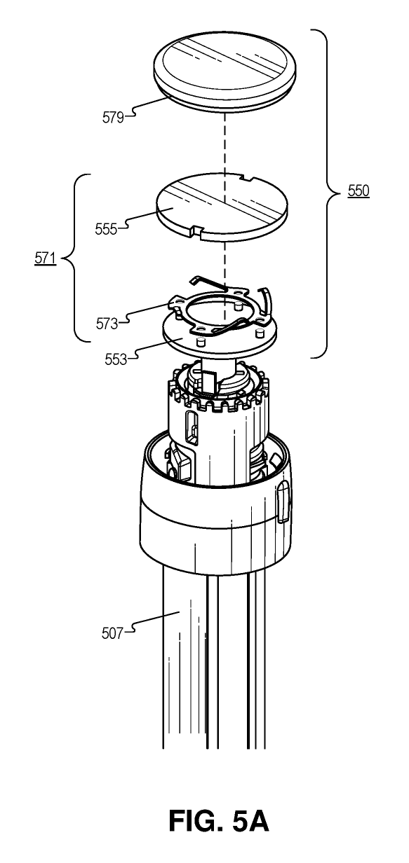

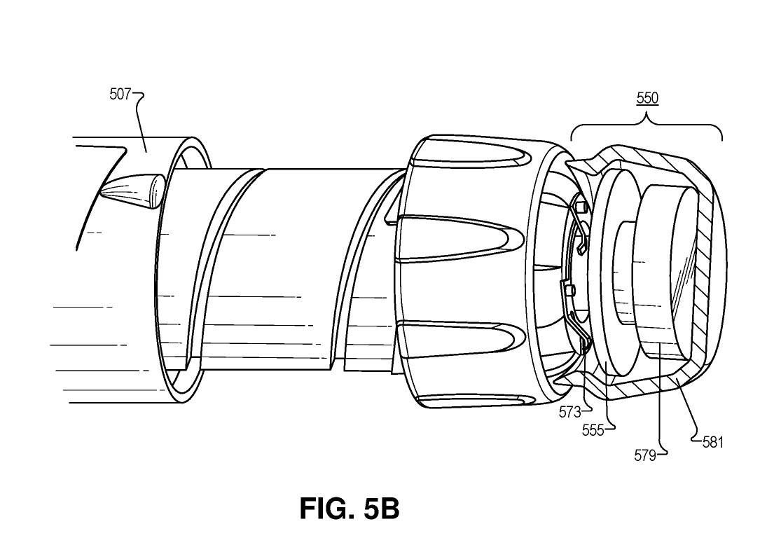

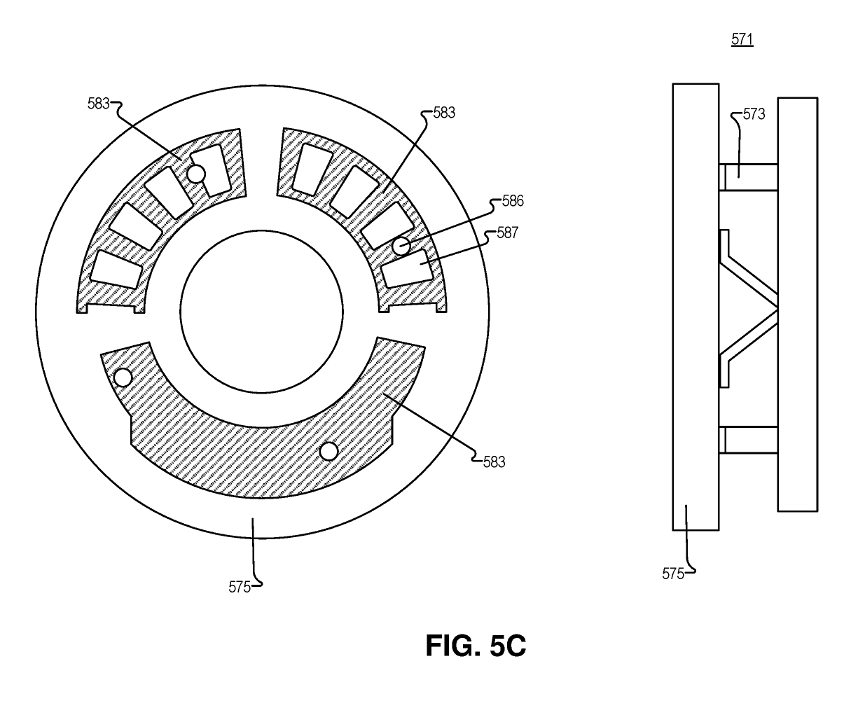

FIG. 5A illustrates an exploded view of a pen button 550 including a dosage measurement system, in accordance with an embodiment of the disclosure. In the depicted embodiment, pen button 550 is attached to a dosage injection mechanism in pen body/housing 507. Pen button 550 includes a mechanical encoder 571 mechanically coupled to the dosage injection mechanism, and at least part of encoder 571 rotates when (or in response to) the fluid/medication is dispensed from the injection pen. Encoder 571 is electrically coupled to a controller within the injection pen, and the controller receives the electrical signal output from encoder 571. The electrical signal from encoder 571 may be representative of the dosage output from the injection pen, and the controller may use this information to calculate the amount of fluid dispensed from the injection pen.

In one embodiment, a pen could contain three concentric column portions (described here as columns A, B, and C) in the dosage injection mechanism, which may rotate independently of each other. When the user is setting the pen's dose, columns A and C may rotate together at the same speed, showing no relative rotation to each other, but columns A and B may show relative rotation with respect to each other. When the user is dispensing insulin, columns A and B may show relative rotation, while A and C do not. Thus, the embodiment depicted here describes a miniaturized encoder 571 that is fabricated within a press button 550. The button 550 may be generally cylindrical and matches the shape of the pre-existing button on the disposable injection pen (e.g., injection pen 101). Multiple form factors can be made to match the multiple commercially available disposable injection pens on the markets. The self-contained press button 550 can then be attached to any disposable drug injection pen to measure and monitor the pen usage. Within the generally cylindrical button assembly may be a power source, encoder 571, controller, radio, and antenna. Pen button 550 automatically collects the volume of each medication injection made with the pen, and also the temperature, time, and date of each injection. The data is stored in the pen's electronics until a smart device (e.g., processing device 121), such as a cellular phone is within radio range, at which time all of the stored data is transferred to the external device. This may happen automatically (without the user needing to initiate the transfer) or manually (with the user initiating transfer). The device may then upload the data to an internet server for further storage and analysis.

Button 550 typically has keyway (see e.g., notches 281 in FIGS. 2A-2D) features that align with the clutch elements of the disposable injection pen. The pre-existing button may be removed and the miniaturized smart button 550 snaps into place using the pre-existing snap features of the disposable injection pen. The snaps (that hold the pre-existing button) and retaining features on smart button 550 also retain the smart button 550 in place. The keyways allow for the self-contained button 550 to measure the relative motion of the dosage injection mechanism in the pen.

A second encoder may be positioned within the disposable pen such that it has elements in contact with two or more rotating portions of the pen's injection mechanism. In many pen designs, there are a plurality of concentric columns that rotate in relation to each other. The relation between column rotation is controlled by clutch mechanisms that are part of the pen's construction. The mechanical function of the pen necessitates the overall arrangement of these clutches and columns. Together, they create an injection pen that conveys force from the user's finger to the rubber stopper of a drug cartridge.

Encoder 571 is attached to elements that show relative rotation (e.g., dosage injection mechanism) when the pen is dispensing insulin. Thus, when setting a dose, there is no relative rotation, and the device does not record any insulin usage. When dispensing insulin, the relative rotation between columns is detected by encoder 571.

As shown, the pen body 507 has a proximal end (opposite the dispensing end) and encoder 571 is disposed in button 550 attached to the proximal end of the pen body 507. In some embodiments, pen button 550 may snap into the back of the pen to mechanically couple to the internal components of the injection pen. This allows pen button 550 to be installed in a multitude of commercially available injection pens. In other words, pen button 550 may be manufactured separately from the rest of the pen components and then subsequently installed by a user, or an end-of-line manufacturer.

As shown, the encoder 571 includes one or more conductive finger elements 573, and circuit board assembly 555 including a metal pattern. The one or more conductive finger elements 573 are in contact with the circuit board assembly 555. In the illustrated embodiment, conductive finger elements 573 are pegged down to a board which may be mechanically coupled to the dosage injection mechanism.

FIG. 5B illustrates an assembled view of the pen button 550 of FIG. 5A with the housing cut away, in accordance with an embodiment of the disclosure. As shown the pen button housing 581 has been cut away to see the assembled components. In the illustrated embodiment, pen button 550 “clips” into the back of the dispensing pen for easy installation.

FIG. 5C illustrates an encoder which may be included in the pen button of FIG. 5A, in accordance with an embodiment of the disclosure. The left-hand side illustrates a face-on view of circuit board assembly 555, and the right-hand side illustrates a side view of the assembled encoder 571. As shown, the one or more conductive finger elements 573 are in contact (dots 586 represent contact points) with metal pattern 583 on circuit board assembly 555. In the depicted embodiment, there are a plurality of conductive finger elements 573 that are electrically coupled to one another. Moreover, metal pattern 583 includes a plurality of subpatterns electrically isolated from one another. As shown, several subpatterns includes metal free sections 587 spaced periodically in the subpatterns.

In the depicted embodiment, encoder 571 is built from a (printed) circuit board assembly 555 (PCBA), and a thin piece of stamped sheet metal forms conductive finger elements 573 that are electrically connected to each other. Metal pattern 583 includes copper that is designed to create quadrature electrical signals as conductive finger elements 573 are rotated across circuit board assembly 555. In order to produce the desired effect, circuit board assembly 555 is attached to one rotating column of the drug injection pen's injection mechanism, and conductive finger elements 573 are attached to another column. The two columns are selected such that they show relative rotation when the pen is dispensing insulin. In the depicted embodiment, the copper foil pattern is designed to work with conductive finger elements 573 that are spaced evenly around the central axis. This is because the large electrode near the bottom of the pattern serves as a common electrode, and the two smaller foil areas serve as the two phases of the quadrature signal. At any given rotation, at least one conductive finger element 573 is in contact with the common electrode. However, the other two foil patterns are spaced 90 degrees apart electrically, such that as the conductive finger elements 573 rotate relative to circuit board assembly 575, the two phases are connected and disconnected from the common electrode separated by 90 degrees. The figure shows an encoder foil pattern with 20 complete cycles (80 quadrature edges) per revolution. This same method can produce encoders with other mechanical resolutions.

In one embodiment, circuit board assembly 555 is attached to the press button of the insulin pen, and when the user applies force to dispense insulin, circuit board assembly 555 moves axially into direct electrical contact with the spring fingers. This is possible because the button engages one of the pen's clutches and is designed to allow some axial movement. Thus, the device can detect when the user is pressing the button even before the device begins to dispense insulin. The gap between the spring fingers and circuit board assembly 555 may be designed so that there is no electrical contact between the two parts when the button is in its resting position. This provides a useful UI feature, and may aid in detection of priming “air” shots.

A mechanical encoder (as described above) uses very little electrical power. The button can incorporate multi-color LED indicators that briefly flash to indicate various states of the device, for example: red—device storage temperature exceeded, insulin expired; green—device active and ready to use; yellow—injection underway, do not withdraw needle yet; and/or blue—data transfer in progress.

The device may be programmed to enter a low power state shortly after final assembly and test at the manufacturing site. It may remain in that state—possibly logging temperature (with a temperature sensor coupled to the controller) and storage time information (with a clock or oscillator coupled to the controller)—until the first use is detected or other event (temperature change, time period elapses, etc.). After this initial activation it will log individual doses and periodically transmit the information to a host receiver (typically a mobile device).

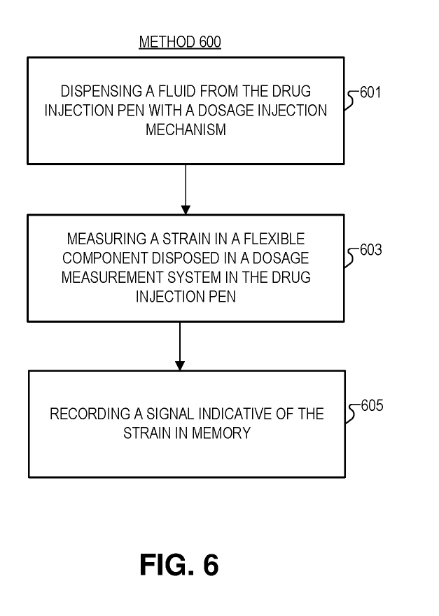

FIG. 6 illustrates a method 600 of dosage measurement, in accordance with an embodiment of the disclosure. One of ordinary skill in the art having the benefit of the present disclosure will appreciate that the blocks of method 600 may occur in any order and even in parallel. Additionally, blocks may be added to, or removed from, method 600 in accordance with the teachings of the present disclosure.

Block 601 shows dispensing a fluid from the drug injection pen with a dosage injection mechanism disposed within the drug injection pen. The dosage injection mechanism (which may include a lead screw) rotates when the fluid is dispensed.

Block 603 illustrates measuring a strain in a flexible component disposed in a dosage measurement system in the drug injection pen, where the strain is imparted in the flexible component in response to the dosage injection mechanism rotating. It is appreciated that, in the depicted embodiment, measuring a strain occurs at the same time as dispensing the fluid (not before or after).

In one embodiment, measuring the strain in the flexible component includes deforming the flexible component with a toothed gear (e.g., toothed gear 253) coupled to the dosage injection mechanism, and the flexible component bends in response to a gear tooth pressing against the flexible component. One or one or more strain sensors that are disposed on the flexible component, and coupled to the controller, may measure the strain and output the strain signal to the controller. In some embodiments, the signal output from the one or more strain sensors may be amplified with amplifiers coupled between the strain sensors and the controller. As shown in embodiments described above, deforming the flexible component may include deforming one or more protrusions extending outward from a circuit board, and the protrusions include the strain sensors.

Block 605 shows recording a signal, indicative of the strain, in memory using a controller coupled to the dosage measurement system to receive the signal. In some embodiments, the controller may then calculate the quantity of the fluid dispensed based, at least in part, on the signal recorded. The controller may transmit the signal to an external processing device, distinct from the drug injection pen, to calculate the quantity of fluid dispensed. Alternatively the controller may locally calculate the quantity of the fluid dispensed.

In some embodiments method 600 may further include a user pressing a pen button disposed on the proximal end of the drug injection pen, opposite a dispensing end. Fluid is dispensed from the drug injection pen in response to the user pressing the button. In these embodiments, the dosage measurement system may be disposed, at least in part, in the button, and a drug delivery control wheel (e.g., drug delivery control wheel 109 of FIG. 1) is disposed between the pen body and the button.

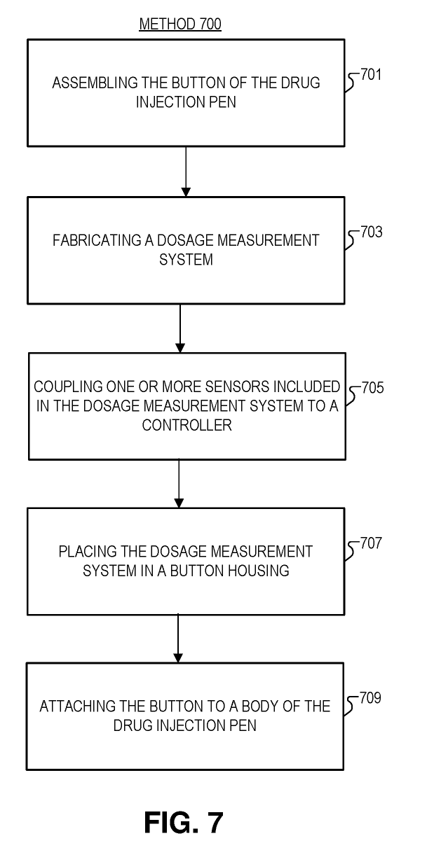

FIG. 7 illustrates a method 700 of fabricating a drug injection pen including a button to measure a dosage dispensed, in accordance with an embodiment of the disclosure. One of ordinary skill in the art having the benefit of the present disclosure will appreciate that the blocks of method 700 may occur in any order and even in parallel. Additionally, blocks may be added to, or removed from, method 700 in accordance with the teachings of the present disclosure.

Block 701 illustrates, assembling the button of the drug injection pen.

Block 703 shows fabricating a dosage measurement system that is part of the button. The dosage measurement system may include a circuit board with a controller coupled to receive a signal indicative of rotational motion of a dosage injection mechanism disposed in the drug injection pen. As stated above, the dosage injection mechanism rotates when the drug injection pen dispenses a fluid.

Block 705 describes coupling one or more sensors included in the dosage measurement system to the controller. In one embodiment, this may be achieved by soldering, or another microelectronic fabrication technique. The one or more sensors may be positioned in the button to measure the rotational motion of the dosage injection mechanism, and output a signal indicative of rotational motion to the controller.

Block 707 illustrates placing the dosage measurement system in a button housing. In this embodiment, the button hosing may be a plastic casing that surrounds the electronics within the button. In some embodiments, the button housing may couple to the injection pen so that it rotates when the pen dispenses a fluid. However, a portion of the button (e.g., the part under the user's thumb) may not rotate with the rest of the housing so the user's fingers do not interfere with drug delivery.

In some embodiments, a toothed gear is placed in the button housing, and the toothed gear is included in the dosage measurement system. The toothed gear is positioned in the button housing to rotate in response to the rotational motion of the dosage injection mechanism, and impart a strain in a flexible component in the dosage measurement system. The one or more sensors are positioned in the button hosing to measure the strain in the flexible component imparted by the toothed gear.

In some embodiments, the flexible component includes one or more protrusions from the circuit board, and coupling the one or more sensors to the controller includes soldering at least one of a capacitive strain sensor, a piezoelectric strain sensor, or a resistive strain sensor to the one or more protrusions. While in other embodiments, coupling one or more sensors to the controller includes coupling an encoder, including one or more conductive finger elements and a metal pattern, to the controller. The one or more conductive finger elements contact the metal pattern when the circuit board assembly rotates relative to the metal pattern in response to the rotational motion.

Block 709 shows attaching the button to a body of the drug injection pen. This may include the button irremovably clipping to the dosage injection mechanism when inserted into a proximal end, opposite the dispensing end, of the drug injection pen (see e.g., FIGS. 2A-2D). In some embodiments, the drug delivery control wheel of the drug injection pen is disposed between a portion of the dosage measurement system and the body, when the button is inserted in the pen. In other words, the drug delivery control wheel is disposed between the pen body and the electronics (e.g., controller, sensors, power supply, transceiver, etc.) in the pen button.

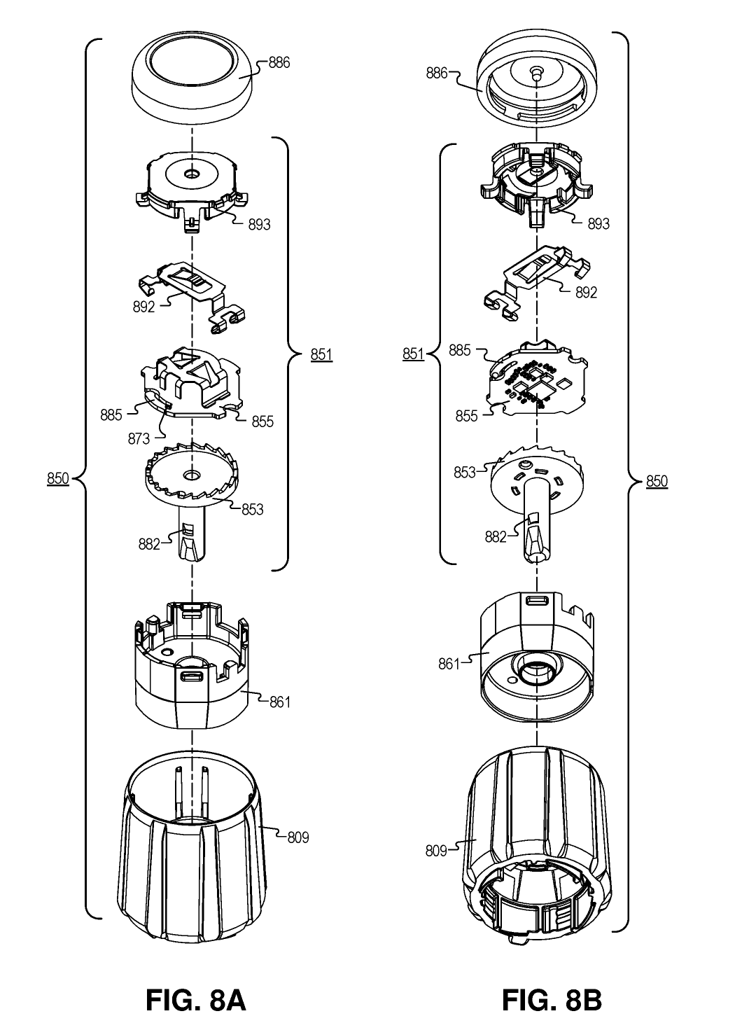

FIGS. 8A-8B illustrate an exploded view of the pen button 850, in accordance with an embodiment of the disclosure. FIGS. 8A and 8B illustrate the same embodiment of pen button 850, but FIG. 8A illustrates an exploded view looking from the top down, and FIG. 8B illustrates an exploded view looking from the bottom up. Pen button 850 includes drug delivery control wheel 809 (also known as a “dial grip”), housing 861, locking tab 882, toothed gear 853, circuit board assembly 855, one or more protrusions 885, one or more strain sensors 873, retaining spring 892, housing clip 893, and spinner 886. As shown, locking tab 882, toothed gear 853, circuit board assembly 855, one or more protrusions 885, one or more strain sensors 873, retaining spring 892, housing clip 893 are disposed in dosage measurement system 851.

In some embodiments, spinner 886 may be made from polybutylene terephthalate (e.g., Celanex 2404MT). Spinner 886 may interact mechanically with (and bear on) housing 861, housing clip 893, and the arm (e.g., center cutout) of retaining spring 892. Housing clip 893 may be made from polycarbonate (e.g., Makrolon 2458). Housing clip 893 may snap fit to housing 861, and housing clip 893 may bear on spinner 886. Toothed gear 853 (e.g., a spindle) may also be made from polycarbonate, and snap into a clutch in the pen. Toothed gear 853 may also bear on housing 861. Housing 861 may be made from polyoxymethylene (e.g., Hostaform MT8F01). And housing 861 may bear on the clutch (e.g., in the pen body), spinner 886, and the linear slide on the drug delivery control wheel 809. Drug delivery control wheel 809 may also be made from polycarbonate, and it interacts with the linear slide on housing 861.

In operation, the components may move together according to the following steps (discussed from a user-fixed reference frame). A user may dial a dose using drug delivery control wheel 809. The user presses down on spinner 886. Spinner 886 presses housing 861 down. Housing 861 presses the clutch inside the pen body down, and the clutch disengages. Drug delivery control wheel 809 and housing 861 will spin with the circuit board assembly 855 as the drugs are dispensed and toothed gear 853 stays rotationally stationary. Drug delivery control wheel 809, housing 861, and circuit board assembly 855 are mechanically coupled to rotate when fluid is dispensed. Tabs on circuit board assembly 855 interact with features on the inside of housing 861 to spin circuit board assembly 855. It is important to note that while dialing a dose, there may be no relative motion between toothed gear 853 and circuit board assembly 855, and that while dispensing, circuit board assembly 855 rotates while toothed gear 853 is fixed to the user-reference frame.

In some embodiments, toothed gear 853 is connected to the clutch (contained in the pen body and included in the dosage injection mechanism)—these parts may not move relative to one another. The clutch is connected to the drive sleeve (also included in the dosage injection mechanism)—which moves axially relative to the clutch with about 1 mm range of motion. The lead screw is threaded into the drive sleeve. If the user has dialed a dose and applies force to button 850, the clutch releases from the numbered sleeve and the lead screw is pushed through a threaded “nut” in the pen body causing the lead screw to advance. When the lead screw advances, it presses on the rubber stopper in the medication vial to dispense medication

In the depicted embodiment, one or more protrusions 885 form a circumferential diving board, and move up/down as the protrusion 885 are deflected by teeth in toothed gear 853. In the depicted embodiment, one or more strain sensors 873 are positioned at the base (e.g., where one or more protrusions 885 meet circuit board assembly 855) of the protrusions 885, where strain is maximized. In the depicted embodiment, strain sensors 873 are positioned on the opposite side of circuit board assembly 855 from toothed gear 853. In this configuration strain sensors 873 operate in compression which—since in some embodiments strain sensors 873 include ceramics (e.g., in the form of piezoelectrics, capacitor dielectrics, or the like)—reduces the probability of failure and degradation. In some embodiments, strain sensors 873 may be placed on components other than circuit board assembly 855.

In the depicted embodiment, instead of triangular ramps (depicted elsewhere), teeth on toothed gear 853 may have a parabolic ramp shape. These ramps may give the integrated circuit in circuit board assembly 855 opportunities to settle when a dose is dialed.

In some embodiments, the device shown in FIG. 8A and 8B may be fabricated according to the following steps. The PCBA on circuit board assembly 855 may be assembled and programed, and the battery is inserted into the metal cage. Toothed gear 853 is inserted into housing 861. Circuit board assembly 855 is inserted into housing 861, and retaining spring 892 is placed on top. Housing clip 893 is snapped into housing 861 above retaining spring 892, and spinner 886 is snapped into housing clip 893. The assembled pen button 850 is then inserted into an assembled pen with a dial grip.

The processes explained above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (“ASIC”) or otherwise.

A tangible machine-readable storage medium includes any mechanism that provides (i.e., stores) information in a non-transitory form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. An apparatus, comprising:
a pen body of a drug injection pen including a dosage injection mechanism that produces a rotational motion when the drug injection pen dispenses a fluid;
a button housing with at least part of a dosage measurement system disposed within the button housing, wherein at least part of the dosage measurement system is coupled to receive the rotational motion from the dosage injection mechanism, the dosage measurement system including:
one or more sensors positioned to output a signal in response to the rotational motion when the drug injection pen dispenses a fluid; and
a controller coupled to the one or more sensors to receive the signal; and
a drug delivery control wheel of the drug injection pen disposed between the body of the drug injection pen and the at least part of the dosage measurement system disposed in the button housing.

2. The apparatus of claim 1, wherein the dosage measurement system irremovably clips into a proximal end, opposite the dispensing end, of the drug injection pen.

3. The apparatus of claim 2, wherein the button housing is positioned to press down on a clutch in the dosage injection mechanism to initiate dispensing the fluid, and wherein the button housing rotates around a longitudinal axis of the drug injection pen when the drug injection pen dispenses the fluid.

4. The apparatus of claim 1, the dosage measurement system further comprising:
one or more strain sensors included in the one or more sensors and disposed on a flexible component of the dosage measurement system to measure a strain in the flexible component when the drug injection pen dispenses a fluid.

5. The apparatus of claim 4, the dosage measurement system further comprising:
a toothed gear; and
a circuit board including the flexible component, wherein the one or more strain sensors are positioned on the circuit board to measure the strain in the circuit board when the circuit board rotates relative to the toothed gear, and teeth on the toothed gear impart strain in the circuit board.

6. The apparatus of claim 5, wherein the circuit board, the button housing, and the drug delivery control wheel all rotate relative to the toothed gear when the drug injection pen dispenses a fluid.

7. The apparatus of claim 4, wherein the one or more strain sensors are disposed at a base of one or more protrusions from the circuit board, and wherein the one or more protrusions flex in response to the toothed gear rotating, and the one or more strain sensors measure the strain in the one or more protrusions, wherein the one or more strain sensors are disposed on a side of the circuit board opposite the toothed gear.

8. The apparatus of claim 4, wherein the strain sensors include at least one of a capacitive strain sensor, a piezoelectric strain sensor, or a resistive strain sensor.

9. The apparatus of claim 1, wherein the one or more sensors includes an encoder disposed in the button housing, wherein the encoder includes one or more conductive finger elements, and a circuit board assembly including a metal pattern, wherein the one or more conductive finger elements contact the metal pattern when the circuit board assembly rotates relative to the one or more conductive finger elements in response to the rotational motion.

10. The apparatus of claim 1, the dosage measurement system further comprising a circuit board coupled to rotate in response to the rotational motion from the dosage injection mechanism, and the circuit board includes one or more protrusions extending outward from the circuit board that are positioned to contact teeth disposed in the button housing.

11. The apparatus of claim 9, wherein the protrusions that extend from the circuit board partially encircle a main portion of the circuit board, and wherein the one or more sensors are disposed proximate to a base of the one or more protrusions to measure a strain in the one or more protrusions.

12. A method of measuring a quantity of fluid dispensed from a drug injection pen, comprising:
measuring a rotational motion of a dosage injection mechanism disposed in the drug injection pen with a dosage measurement system disposed in a button housing attached to a proximal end of the drug injection pen, wherein at least part of the dosage measurement system is mechanically coupled to the dosage injection mechanism, and wherein a drug delivery control wheel of the drug injection pen is disposed between a portion of the dosage measurement system in the button housing and a body of the drug injection pen;
receiving a signal, output from the dosage measurement system in response to the rotational motion, with a controller coupled to the dosage measurement system; and
recording the signal output from the dosage measurement system in a memory.

13. The method of claim 12, wherein measuring the rotational motion of the dosage injection mechanism includes:
deforming a flexible component in the dosage measurement system with a toothed gear, wherein the flexible component bends in response to a gear tooth pressing against the flexible component; and
measuring a strain with one or more strain sensors disposed on the flexible component and coupled to the controller to output the signal to the controller.

14. The method of claim 13, wherein measuring the strain includes using at least one of a capacitive strain sensor, a piezoelectric strain sensor, or a resistive strain sensor.

15. The method of claim 12, wherein measuring the rotational motion of the dosage injection mechanism includes using a circuit board coupled to rotate in response to the rotational motion from the dosage injection mechanism, and the circuit board includes one or more protrusions extending outward from the circuit board that are positioned to contact teeth disposed in the button housing when the circuit board rotates.

16. The method of claim 15, wherein measuring the strain with the one or more strain sensors includes measuring compression or a tension at a base of the one or more protrusions, wherein the one or more protrusions encircle the circuit board at least in part or extend outward from the circuit board radially.

17. The method of claim 12, wherein measuring the rotational motion of the dosage injection mechanism includes using an encoder disposed in the button housing, wherein the encoder includes one or more conductive finger elements, and a circuit board assembly including a metal pattern, wherein the one or more conductive finger elements contact the metal pattern when the circuit board assembly rotates relative to the one or more conductive finger elements in response to the rotational motion.

18. A method of manufacturing a drug injection pen including a button to measure a quantity of fluid dispensed from the drug injection pen, comprising:
assembling the button of the drug injection pen, including:
fabricating a dosage measurement system including a circuit board with a controller coupled to receive a signal indicative of rotational motion of a dosage injection mechanism disposed in the drug injection pen;
coupling one or more sensors included in the dosage measurement system to the controller, wherein the one or more sensors are positioned in the button to measure the rotational motion of the dosage injection mechanism and output the signal to the controller; and
placing the dosage measurement system in a button housing; and
attaching the button to the drug injection pen, wherein the button irremovably clips to the drug injection pen when inserted into a proximal end, opposite the dispensing end, of the drug injection pen, and wherein a drug delivery control wheel of the drug injection pen is disposed between a portion of the dosage measurement system and a body of the drug injection pen.

19. The method of claim 18, further comprising:
positioning a toothed gear, included in the dosage measurement system, in the button housing to impart a strain in a flexible component in the dosage measurement system, and wherein the one or more sensors are positioned to measure the strain in the flexible component.

20. The method of claim 19, wherein the flexible component includes one or more protrusions from the circuit board, and wherein coupling the one or more sensors to the controller includes soldering at least one of a capacitive strain sensor, a piezoelectric strain sensor, or a resistive strain sensor to the one or more protrusions.

21. The method of claim 18, wherein coupling one or more sensors to the controller includes:
coupling an encoder, including one or more conductive finger elements and a metal pattern, to the controller, wherein the one or more conductive finger elements contact the metal pattern when the one or more conductive finger elements rotate relative to the metal pattern in response to the rotational motion.

22. The method of claim 18, wherein fabricating a dosage injection mechanism includes:
forming one or more protrusions extending outward from, or encircling at least in part, the circuit board that are positioned to contact teeth disposed in the button when the circuit board rotates, and wherein coupling the one or more sensors to the controller includes soldering the one or more sensors to the one or more protrusions.
